(19) United States
(12) Patent Application Publication
(10) Pub. No.: US 2026/0060916 A1
(43) Pub. Date: Mar. 5, 2026

(54) OIL-BASED COSMETIC

(71) Applicant: Tombow Pencil Co., Ltd., Tokyo (JP)

(72) Inventors: Noriaki OZAKI, Tokyo (JP); Yoshikazu SOYAMA, Tokyo (JP)

(73) Assignee: Tombow Pencil Co., Ltd., Tokyo (JP)

(21) Appl. No.: 19/311,783

(22) Filed: Aug. 27, 2025

(30) Foreign Application Priority Data
Sep. 4, 2024 (JP) 2024-152328

(51) Int. Cl. A61K 8/92; A61Q 1/02

(52) U.S. Cl. CPC A61K 8/922; A61Q 1/02; A61K 2800/34; A61K 2800/43

(57) ABSTRACT

Provided in an oil-based cosmetic containing: (a) a non-volatile oil agent having a kinematic viscosity at 25° C. of 4 mm²/s to 20 mm²/s and a boiling point at 1 atm of lower than 290° C.; (b) a spherical particle; (c) a film-forming agent; and (d) a color material, in which a mass ratio of a content of (b) the spherical particle to a content of (a) the non-volatile oil agent is 1.0 to 3.5.

This application is based on and claims the benefit of priority from Japanese Patent Application No. 2024-152328 filed on 4 Sep. 2024, the contents of which is incorporated herein by reference.

### BACKGROUND OF THE INVENTION

### Field of the Invention

The present disclosure relates to an oil-based cosmetic.

### Related Art

The oil-based cosmetic is of a form of a cosmetic widely used for foundation, eye shadow, and the like, and various functions and feeling of use can be realized by changing a type or a blending amount of an oil agent and powder to be blended in the cosmetic.

The present inventors have previously proposed, in Patent Document 1, an oil-based powder foundation containing spherical particles, titanium dioxide, a film-forming agent, a volatile oil agent, and a surfactant. The oil-based powder foundation described in Patent Document 1 has good spreadability, which is a feature of an oil-based product, and also has a function of rapidly changing to a powder feeling after application, and is excellent in makeup lasting property (sebum resistance and sweat resistance).
Patent Document 1: Japanese Unexamined Patent Application, Publication No. 2022-176122

### SUMMARY OF THE INVENTION

Since the oil-based powder foundation described in Patent Document 1 contains a relatively large amount of volatile oil agent, an airtight container is required during storage. In this regard, in a case of a composition containing no volatile liquid or having a low content ratio of the volatile liquid, various containers other than the airtight container can be used, and a change in composition due to drying can be prevented as much as possible.

Therefore, an object of the present disclosure is to provide a novel oil-based cosmetic that is excellent in spreadability at the time of application, is rapidly changed to a powder feeling after application, and is excellent in makeup lasting property even when a volatile liquid is not essential.

Specific means for solving the above problems include the following embodiments.

<1> An oil-based cosmetic containing:
(a) a non-volatile oil agent having a kinematic viscosity at 25° C. of 4 mm²/s to 20 mm²/s and a boiling point at 1 atm of 290° C. or higher;
(b) a spherical particle;
(c) a film-forming agent; and
(d) a color material,
in which a mass ratio of a content of (b) the spherical particle to a content of (a) the non-volatile oil agent being 1.0 to 3.5.

<2> The oil-based cosmetic as described in aspect <1>, in which a content ratio of a volatile liquid having a boiling point at 1 atm of lower than 290° C. is less than 5 mass %.

<3> The oil-based cosmetic as described in aspect <1> or <2>, in which a content ratio of (a) the non-volatile oil agent is 15 mass % to 50 mass %.

<4> The oil-based cosmetic as described in any of aspects <1> to <3>, in which a content ratio of (b) the spherical particle is 30 mass % to 65 mass %.

<5> The oil-based cosmetic as described in any of aspects <1> to <4>, further containing: (e) a wax.

<6> The oil-based cosmetic as described in any of aspects <1> to <5>, in which the oil-based cosmetic does not contain a silicone compound.

<7> The oil-based cosmetic as described in any of aspects <1> to <6>, in which a natural origin index (excluding water) calculated in accordance with ISO 16128 is 90% or more.

<8> The oil-based cosmetic as described in any of aspects <1> to <7>, which is a cosmetic for foundation, eye shadow, eyebrow, cheek, or base.

According to the present disclosure, it is possible to provide a novel oil-based cosmetic that is excellent in spreadability at the time of application, is rapidly changed to a powder feeling after application, and is excellent in makeup lasting property even when a volatile liquid is not essential.

### DETAILED DESCRIPTION OF THE INVENTION

Hereinafter, specific embodiments of the present disclosure will be described in detail. In the present description, unless otherwise specified, a notation “x to y” using numerical values x and y means “x or more and y or less”. In such a notation, when a unit is added to only the numerical value y, the unit is also applied to the numerical value x.

### <Oil-Based Cosmetic>

The oil-based cosmetic according to the present embodiment contains: (a) a non-volatile oil agent having a kinematic viscosity at 25° C. of 4 mm²/s to 20 mm²/s and a boiling point at 1 atm of 290° C. or higher (hereinafter, also referred to as “component (a)”); (b) a spherical particle (hereinafter, also referred to as “component (b)”); (c) a film-forming agent (hereinafter, also referred to as “component (c)”); and (d) a color material (hereinafter, also referred to as “component (d)”), and a mass ratio of a content of the component (b) to a content of the component (a) is 1.0 to 3.5.

The oil-based cosmetic according to the present embodiment is excellent in spreadability at the time of application, is rapidly changed to a powder feeling after application, and is excellent in makeup lasting property even when a volatile liquid is not essential. A reason for this is unclear, but is presumed as follows. That is, at the time of application, excellent spreadability is realized by the non-volatile oil agent having a low viscosity, and after application, a powder feeling is obtained by the spherical particle. Further, since the spherical particle on a skin surface is fixed by the film-forming agent, excellent makeup lasting property is realized.

Hereinafter, each of the components contained in the oil-based cosmetic according to the present embodiment will be described in detail. Each of the following components may be used alone, or may be used in combination of two or more kinds thereof.

### [(a) Non-Volatile Oil Agent]

In the present description, the term “non-volatile” means that the boiling point at 1 atm is 290° C. or higher.

A non-volatile oil agent as the component (a) has a kinematic viscosity at 25° C. of 4 mm²/s to 20 mm²/s (4 cs to 20 cs). When the kinematic viscosity of the non-volatile oil agent at 25° C. is 4 mm²/s or more, good spreadability, which is a feature of an oil-based product, tends to be easily felt. In addition, when the kinematic viscosity of the non-volatile oil agent at 25° C. is 20 mm²/s or less, the spreadability at the time of application is improved, and a good powder feeling tends to be obtained after application. The kinematic viscosity of the non-volatile oil agent at 25° C. is preferably 4 mm²/s to 18 mm²/s, and more preferably 4 mm²/s to 15 mm²/s. The kinematic viscosity is a measured value at 25° C. using a B-type viscometer.

The non-volatile oil agent is not particularly limited as long as it has a kinematic viscosity at 25° C. of 4 mm²/s to 20 mm²/s and can be used for cosmetics. Examples of the non-volatile oil agent include ester oils such as dicaprylyl carbonate, coco-caprylate/caprate, heptyl undecylenate, hydrogenated ethylhexyl olivate/hydrogenated olive oil unsaponifiables, ethyl isostearate, and hexyl laurate; and hydrocarbon oils such as C15-19 alkane and liquid paraffin. Among them, a plant-derived oil agent is preferable from the viewpoint of reducing an environmental load.

As the non-volatile oil agent, a commercially available product can also be used. Examples of a commercially available product of the dicaprylyl carbonate include Cetiol CC (manufactured by BASF). Examples of a commercially available product of the coco-caprylate/caprate include Liponate CCC MB (manufactured by Vantage Specialty Ingredients, Inc.) and Cetiol C5C (manufactured by BASF). Examples of a commercially available product of the heptyl undecylenate include LexFeel Natural (manufactured by INOLEX). Examples of a commercially available product of the hydrogenated ethylhexyl olivate/hydrogenated olive oil unsaponifiables include Natura-Tec Plantsil (manufactured by H. Holstein Co., Ltd.) and Vegetable Alternative to Silicone (VAS) (manufactured by Kowa Company, Ltd.). Examples of a commercially available product of the ethyl isostearate include EIS-V (manufactured by KOKYU ALCOHOL KOGYO CO., LTD.). Examples of a commercially available product of the hexyl laurate include KAK HL (manufactured by KOKYU ALCOHOL KOGYO CO., LTD.). Examples of a commercially available product of the C15-19 alkane include Jeechem NDA-5 (manufactured by Jeen International Corporation) and EMOSMART L19 (manufactured by SEPPIC S.A.). Examples of a commercially available product of the liquid paraffin include liquid paraffin No. 50S (manufactured by Sanko Chemical Industry Co., Ltd.).

A content ratio of the component (a) is preferably 15 mass % to 50 mass %, more preferably 18 mass % to 45 mass %, and further preferably 20 mass % to 40 mass %.

### [(b) Spherical Particle]

In the present description, the term “spherical” includes not only a true spherical shape but also a substantially spherical shape, an elliptical spherical shape, and the like, and means a shape in which a ratio (aspect ratio) of a major axis to a minor axis is 1.0 to 1.5, and preferably 1.0 to 1.2. A spherical particle as the component (b) may have a smooth surface, may have pores on the surface, or may have minute irregularities on the surface.

A volume-weighted mean diameter of the spherical particle is preferably 5 μm to 30 μm, more preferably 6 μm to 25 μm, and further preferably 7 μm to 20 μm. When the volume-weighted mean diameter of the spherical particle falls within the above range, the oil-based cosmetic according to the present embodiment tends to have good feeling of use, finish, and soft focus function (gradation function for making pores and the like less noticeable). The volume-weighted mean diameter is a particle diameter (median diameter) at which a cumulation reaches 50% in a case of a cumulative volume distribution curve is drawn from a small diameter side in particle size distribution measured by a laser diffraction/scattering method.

A material of the spherical particle is preferably selected from the viewpoints of dispersibility (wettability of spherical particle) and usability in the oil-based cosmetic. Examples of the spherical particle include cellulose powder, cellulose acetate powder, calcium carbonate powder, silica powder, and urethane powder. Among them, plant-derived spherical particles such as cellulose and cellulose acetate are preferable from the viewpoint of reducing an environmental load.

As the spherical particle, a commercially available product can also be used. Examples of a commercially available product of the cellulose powder include CELLULOBEADS D-10 and CELLULOBEADS D-5 (all manufactured by DAITO CHEMICAL CO., LTD.), Celluflow C-25 (manufactured by JNC Corporation), RUBLALEAF KCX (manufactured by Dainichiseika Color & Chemicals Mfg. Co., Ltd.), and GE-800 (manufactured by TOSHIKI PIGMENT CO., LTD.). Examples of a commercially available product of the cellulose acetate powder include BELLOCEA S7 (manufactured by Daicel Corporation) and Celluflow TA-25 (manufactured by JNC Corporation). Examples of a commercially available product of the calcium carbonate powder include SCS-M5 (manufactured by Sakai Chemical Industry Co., Ltd.) and NL-QC10 (manufactured by NEW LIME CO., LTD.). Examples of a commercially available product of the silica powder include SB-700 (manufactured by Miyoshi Kasei, Inc.). Examples of a commercially available product of the urethane powder include TP Powder D-400 and D-800 (all manufactured by TOSHIKI PIGMENT CO., LTD.).

A content ratio of the component (b) is preferably 30 mass % to 65 mass %, and more preferably 35 mass % to 60 mass %.

A mass ratio of the content of the component (b) to the content of the component (a) is 1.0 to 3.5. When the content of the component (b) is 1.0 time or more the content of the component (a), a good powder feeling tends to be obtained after application. In addition, when the content of the component (b) is 3.5 times or less the content of the component (a), the spreadability at the time of application tends to be good and the makeup lasting property after application tends to be good. The mass ratio of the content of the component (b) to the content of the component (a) is preferably 1.1 to 3.0, and more preferably 1.1 to 2.3.

### [(c) Film-Forming Agent]

A film-forming agent as the component (c) exhibits a paste state or a solid state at 25° C., and has a property of dissolving in the non-volatile oil agent as the component (a) to form a film on a skin.

Examples of the film-forming agent include rosin-based resins (pentaerythrityl hydrogenated rosinate, pentaerythrityl rosinate, glyceryl rosinate, and the like); acrylic resins having an acrylic polymer as a main chain and having a hydrophobic group in a side chain; natural ester resins (candelilla wax extract and carnauba wax extract); sugar fatty acid esters (dextrin isostearate and the like); modified starch; isobutene-based resins (polyvinyl isobutyl ether, polyisobutylene, and the like); and vinyl acetate-based resins. Among them, the dextrin isostearate and the candelilla wax extract are preferable from the viewpoints of makeup lasting property, reduction in environmental load, and the like.

The film-forming agent is preferably used by dissolving in a solvent from the viewpoint of uniformly spreading the film-forming agent during use. Examples of the solvent include triethylhexanoin.

As the film-forming agent, a commercially available product can also be used. For example, examples of a commercially available product of the pentaerythrityl hydrogenated rosinate include Ester Gum HP (manufactured by Arakawa Chemical Industries, Ltd.). Examples of a commercially available product of the candelilla wax extract include candelilla resin E-1 (manufactured by Japan Natural Products Co., Ltd.). Examples of a commercially available product of the carnauba wax extract include TOWAX-1B4 (manufactured by TOA KASEI CO., LTD.). Examples of a commercially available product of the dextrin isostearate include Unifilma HVY and Unifilma Liquid TEH (triethylhexanoin solution having a concentration of 75 mass %) (all manufactured by Chiba Flour Milling Co., Ltd.).

A content ratio of the component (c) is preferably 0.1 mass % to 15 mass %, more preferably 0.5 mass % to 12 mass %, and further preferably 1 mass % to 10 mass %.

### [(d) Color Material]

A color material as the component (d) is not particularly limited as long as it can be used in cosmetics. The color material is not limited to a chromatic material, and may be an achromatic material, a pearling agent, a brightening agent, or the like. Examples of the color material include inorganic white pigments such as titanium dioxide and zinc oxide; inorganic red pigments such as iron oxide (red iron oxide) and iron titanate; inorganic brown pigments such as y-iron oxide; inorganic yellow pigments such as yellow iron oxide and loess; inorganic black pigments such as black iron oxide and low-order titanium oxide; inorganic violet pigments such as manganese violet and cobalt violet; inorganic green pigments such as chromium oxide, chromium hydroxide, and cobalt titanate; inorganic blue pigments such as ultramarine blue and Prussian blue; carbon-based black pigments such as carbon black and graphite; metal powder such as aluminum powder, gold powder, silver powder, and copper powder; pearling agents such as titanium oxide-coated mica and iron oxide-titanium oxide-coated mica; brightening agents such as boron nitride and mica; organic pigments such as Red No. 104, Red No. 201, Red No. 202, Red No. 218, Red No. 219, Red No. 220, Red No. 223, Red No. 225, Red No. 226, Red No. 227, Red No. 228, Red No. 504, Orange No. 201, Orange No. 205, Yellow No. 4, Yellow No. 5, Yellow No. 201, Yellow No. 203, Yellow No. 204, Yellow No. 403, Blue No. 1, Blue No. 404, Green No. 3, Green No. 201, Green No. 202, Green No. 204, and purple No. 201; and natural colorants such as β-carotene, a cotinyl pigment, a red cabbage pigment, riboflavin, crocin, anthraquinone, canthaxanthin, and a safflower pigment.

The color material may be subjected to a surface treatment in order to enhance wettability to the non-volatile oil agent. Examples of a surface treatment agent include a fluorine compound, a silane coupling agent, a metal soap, a fatty acid, and a plant-derived ester. Among them, the plant-derived ester is preferable, and polyglyceryl-2 tetraisostearate is more preferable, from the viewpoint of reducing an environmental load.

A content ratio of the component (d) is preferably 0.1 mass % to 30 mass %, more preferably 1 mass % to 25 mass %, and further preferably 5 mass % to 20 mass %.

### [(e) Wax]

The oil-based cosmetic according to the present embodiment may contain (e) a wax (hereinafter, also referred to as “component (e)”) in order to obtain, for example, a solid type cosmetic.

Examples of the wax include natural waxes such as sunflower wax, carnauba wax, candelilla wax, beeswax, Japan wax, and rice bran wax; mineral waxes such as a polyethylene wax, a paraffin wax, a microcrystalline wax, and ceresin; saturated fatty acids such as myristic acid, palmitic acid, stearic acid, arachic acid, and behenic acid; higher fatty acid esters such as cetyl palmitate; and higher alcohols such as cetanol, stearyl alcohol, and behenyl alcohol. Among them, a plant-derived wax is preferable from the viewpoint of reducing an environmental load.

In a case where the oil-based cosmetic according to the present embodiment contains the component (e), a content ratio thereof is preferably 0.1 mass % to 5 mass %, more preferably 0.5 mass % to 4 mass %, and further preferably 1 mass % to 3.5 mass %.

### [(f) Surfactant]

The oil-based cosmetic according to the present embodiment may contain (f) a surfactant (hereinafter, also referred to as “component (f)”) in order to enhance wettability of the component (b) and the component (d) to the non-volatile oil agent.

A surfactant as the component (f) is preferably a nonionic surfactant having a hydrophilic-lypophilic balance (HLB) value of 10 or less, and more preferably a nonionic surfactant having an HLB value of 6 or less. The HLB value is a value indicating a balance between hydrophilicity and lipophilicity, and is determined by a Griffin formula. In a case where two or more kinds of surfactants are used, an arithmetic average value of values obtained by multiplying the HLB value of each surfactant by a blending ratio of each surfactant is defined as the HLB value.

Examples of the nonionic surfactant having an HLB value of 10 or less include sorbitan esters such as sorbitan monoisostearate, sorbitan monooleate, sorbitan sesquiisostearate, and sorbitan sesquioleate; diglyceryl esters such as diglyceryl monoisostearate, and diglyceryl monooleate; and sucrose fatty acid esters, polyoxyethylene alkyl ethers, and polyoxyethylene hardened castor oils. Among them, a plant-derived nonionic surfactant is preferable from the viewpoint of reducing an environmental load.

In a case where the oil-based cosmetic according to the present embodiment contains the component (f), a content ratio thereof is preferably 0.1 mass % to 5 mass %, more preferably 0.3 mass % to 4 mass %, and further preferably 0.5 mass % to 3 mass %.

### [Other Components]

### (Volatile Liquid)

The oil-based cosmetic according to the present embodiment does not necessarily require a volatile liquid, but may contain a volatile liquid. In the present description, the term “volatile” means that the boiling point at 1 atm is lower than 290° C. Examples of the volatile liquid include C9-12 alkane, isododecane, n-tridecane, and water.

A content ratio of the volatile liquid is preferably less than 5 mass %, more preferably 3 mass % or less, further preferably 1 mass& or less, and may be 0 mass % (that is, no volatile liquid is contained).

### (Organic Modified Bentonite)

The oil-based cosmetic according to the present embodiment may contain an organic modified bentonite for adjusting the viscosity or the like. The organic modified bentonite is a kind of colloidal hydrated aluminum silicate having a three-layer structure, and is typically a clay mineral modified with a quaternary ammonium salt type cationic surfactant. Examples of the organic modified bentonite include dimethyldistearylammonium hectorite (disteardimonium hectorite), dimethylalkylammonium hectorite, benzyldimethylstearylammonium hectorite, and distearyldimethylammonium chloride-treated aluminum magnesium.

### (Others)

The oil-based cosmetic according to the present embodiment may contain, in addition to the components described above, powder (barium sulfate or the like) other than the component (b) and the component (d), an ultraviolet absorber, an antioxidant, an antiseptic, and the like. In addition, as long as the effect of the present disclosure is not impaired, the oil-based cosmetic may contain a non-volatile oil agent (having a kinematic viscosity at 25° C. outside the range of 4 mm²/s to 20 mm²/s) other than the component (a).

### [Method for Preparing Oil-Based Cosmetic]

A method for preparing an oil-based cosmetic according to the present embodiment is not particularly limited, and a known method for preparing an oil-based cosmetic can be adopted. An example of the preparation method is as follows. First, the component (a) and the component (c), as well as the component (e), the component (f), and other components blended as necessary are heated to 80° C. to 90° C. and dissolved. Next, the component (b) and the component (d), as well as other components blended as necessary are added to the obtained liquid and stirred. After stirring, the mixture is allowed to stand at 80° C. to 90° C. for defoaming, filled in a container, and cooled.

The oil-based cosmetic according to the present embodiment preferably does not contain a silicone compound from the viewpoint of reducing an environmental load and the like. In addition, in the oil-based cosmetic according to the present embodiment, from the viewpoint of reducing an environmental load and the like, a natural origin index (excluding water) calculated in accordance with ISO 16128 (hereinafter, simply abbreviated as “natural origin index”) is preferably 90% or more, more preferably 95% or more, and further preferably 98% or more.

### [Application of Oil-Based Cosmetic]

The oil-based cosmetic according to the present embodiment has good spreadability, which is a feature of an oil-based product, and also has a function of rapidly changing to a powder feeling after application, and is excellent in makeup lasting property (sebum resistance and sweat resistance). Therefore, the oil-based cosmetic according to the present embodiment can be suitably used as a cosmetic for foundation (solid type or liquid type), eye shadow, eyebrow, cheek, base, or the like.

### EXAMPLES

Hereinafter, the present disclosure will be described more specifically with reference to Examples, but the present disclosure is not limited by these Examples.

### Examples 1 to 11 and Comparative Examples 1 to 5

Oil-based cosmetics of Examples 1 to 11 and Comparative Examples 1 to 5 were prepared as follows. First, among respective components shown in the following Tables 1 to 5, a component (a) or an oil agent, a component (c), a component

(e), and a component (f) were mixed, heated to 80° C. to 90° C., and dissolved. Next, a component (b), a component (d), and other components were added to the obtained liquid and stirred. After stirring, the mixture was allowed to stand at 80° C. to 90° C. for defoaming, filled in a container, and cooled. Numerical values in a column of each component in Tables 1 to 5 indicate a blending ratio (unit:mass %) of the component, and a blank indicates that the component is not blended. In addition, all of kinematic viscosities in Tables 1 to 5 are values at 25° C.

### <Evaluation>

The oil-based cosmetics of Examples 1 to 11 and Comparative Examples 1 to 5 were subjected to an actual use test by six specialists to evaluate four items of “spreadability”, “powder feeling”, “finish”, and “makeup lasting property”. Scoring was performed with evaluation points of 1 to 5 shown below, and an average value (obtained by rounding off second decimal point) of those from the six specialists was evaluated according to an evaluation criteria of 4 levels of A to D shown below. Results are shown in Tables 1 to 5 below.

### (Evaluation Points)

5: Very good
4: Good
3: Normal
2: Slightly inferior
1: Inferior

### (Evaluation Criteria)

A: An average value of the evaluation points is 4.1 or more
B: An average value of the evaluation points is 3.1 to 4.0
C: An average value of the evaluation points is 2.1 to 3.0
D: An average value of the evaluation points is 2.0 or less

TABLE 1

| | | Natural | Kinematic | | | | | |
|---|---|---|---|---|---|---|---|---|
| | | origin | viscosity | Example | | | | |
| Component (mass %) | | index | (mm²/s) | 1 | 2 | 3 | 4 | 5 |
| (a) | Dicaprylyl carbonate *1 | 1.00 | 6 | 25.0 | | | | |
| | Coco-csprylate/caprate *2 | 1.00 | 6 | | 28.0 | | | 24.5 |
| | Ethyl isostearate *3 | 1.00 | 8 | | | 22.0 | | |
| | Hexyl laurate *4 | 1.00 | 5 | | | | | |
| | Liquid paraffin *5 | 0.00 | 11 | | | | 15.0 | |
| Oil | Squalane *6 | 1.00 | 30 | | | | | |
| agent | Jojoba oil *7 | 1.00 | 40 | | | | | |
| (b) | Cellulose powder (14 μm) *8 | 1.00 | | 48.4 | | 43.5 | | 41.5 |
| | Cellulose acetate powder (7 μm) *9 | 1.00 | | | 47.9 | | | |
| | Urethane powder (7 μm) *10 | 0.00 | | | | | 42.5 | |
| (c) | Dextrin isostearate *11 | 1.00 | | 4.0 | 3.0 | | | 4.0 |
| | Candelilla wax extract *12 | 1.00 | | | | 4.0 | | |
| | Trimethylsiloxysilicic acid/dimethicone mixture *13 | 0.00 | Dimethicone: 6 | | | | 13.0 | |
| (d) | Polyglyceryl-2 tetraisostearate-treated titanium dioxide *14 | 0.97 | | 16.0 | 16.0 | 16.0 | | 16.0 |
| | Polyglyceryl-2 tetraisostearate-treated red iron oxide *15 | 1.00 | | 1.5 | 1.5 | 1.5 | | 1.5 |
| | Polyglyceryl-2 tetraisostearate-treated yellow iron oxide *16 | 1.00 | | 0.5 | 0.5 | 0.5 | | 0.5 |
| | Polyglyceryl-2 tatraisostearate-treated black iron oxide *17 | 1.00 | | 0.1 | 0.1 | 0.1 | | 0.1 |
| | Silicone-treated titanium dioxide *18 | 0.98 | | | | | 16.0 | |
| | Silicone-treated yellow iron oxide *19 | 0.97 | | | | | 1.5 | |
| | Silicone-treated red iron oxide *20 | 0.97 | | | | | 0.5 | |
| | Silicone-treated black iron oxide *21 | 0.98 | | | | | 0.1 | |
| (e) | Sunflower wax *22 | 1.00 | | 2.0 | 1.0 | 3.0 | | |
| | Carnauba wax *23 | 1.00 | | 0.5 | 1.0 | 0.5 | | |
| | Ceresin *24 | 0.00 | | | | | 3.0 | |
| (f) | Sorbitan sesquioleate *25 | 1.00 | | | 1.0 | | 1.5 | 1.0 |
| Others | C9-12 alkane *26 | 1.00 | 1.8 | | | | | 3.0 |
| | Isododecane *27 | 0.00 | | | | 2.0 | | |
| | Barium sulfate *28 | 1.00 | | 2.0 | | 6.9 | 6.9 | 6.9 |
| | Disteardimonium hectorite *29 | 0.98 | | | | | | 1.0 |

| Total | | 100.0 | 100.0 | 100.0 | 100.0 | 100.0 |
|---|---|---|---|---|---|---|
| Mass ratio of component (b) to component (a) or oil agent | | 1.9 | 1.7 | 2.0 | 2.1 | 1.7 |
| Presence/absence of silicone compound | | Absent | Absent | Absent | Present | Absent |
| Natural origin index (%) | | >99 | >99 | >97 | 26 | >99 |
| Evaluation | Spreadability | A | A | A | A | A |
| result | Powder feeling | A | A | A | B | A |
| | Finish | A | A | A | A | A |
| | Makeup lasting property | A | A | A | A | A |

TABLE 2

| | | Natural | Kinematic | Comparative | |
|---|---|---|---|---|---|
| | | origin | viscosity | Exemple | |
| Component (mass %) | | index | (mm²/s) | 1 | 2 |
| (a) | Dicaprylyl carbonate *1 | 1.00 | 6 | | |
| | Coco-caprylate/caprate *2 | 1.00 | 6 | | |
| | Ethyl isostearate *3 | 1.00 | 8 | | |
| | Hexyl laurate *4 | 1.00 | 5 | | |
| | Liquid paraffin *5 | 0.00 | 11 | | |
| Oil | Squalane *6 | 1.00 | 30 | 28.0 | |
| agent | Jojoba oil *7 | 1.00 | 40 | | 25.0 |
| (b) | Cellulose powder (14 μm) *8 | 1.00 | | 48.4 | 49.9 |
| | Cellulose acetate powder (7 μm) *9 | 1.00 | | | |
| | Urethane powder (7 μm) *10 | 0.00 | | | |
| (c) | Dextrin isostearate *11 | 1.00 | | 1.0 | 4.0 |
| | Candelilla wax extract *12 | 1.00 | | | |
| | Trimethylsiloxysilicic acid/dimethicone mixture *13 | 0.00 | Dimethicone: 6 | | |
| (d) | Polyglyceryl-2 tetraisostearate-treated titanium dioxide *14 | 0.97 | | 16.0 | 16.0 |
| | Polyglyceryl-2 tetraisostearate-treated red iron oxide *15 | 1.00 | | 1.5 | 1.5 |
| | Polyglyceryl-2 tetraisostearate-treated yellow iron oxide *16 | 1.00 | | 0.5 | 0.5 |
| | Polyglyceryl-2 tetraisostearate-treated black iron oxide *17 | 1.00 | | 0.1 | 0.1 |
| | Silicone-treated titanium dioxide *18 | 0.98 | | | |
| | Silicone-treated yellow iron oxide *19 | 0.97 | | | |
| | Silicone-treated red iron oxide *20 | 0.97 | | | |
| | Silicone-treated black iron oxide *21 | 0.98 | | | |
| (e) | Sunflower wax *22 | 1.00 | | 3.0 | 2.0 |
| | Carnauba wax *23 | 1.00 | | 0.5 | |
| | Ceresin *24 | 0.00 | | | |
| (f) | Sorbitan sesquioleate *25 | 1.00 | | 1.0 | 1.0 |
| Others | C9-12 alkane *26 | 1.00 | 1.8 | | |
| | Isododecane *27 | 0.00 | | | |
| | Barium sulfate *28 | 1.00 | | | |
| | Disteardimonium hectorite *29 | 0.98 | | | |

| Total | | 100.0 | 100.0 |
|---|---|---|---|
| Mass ratio of component (b) to component (a) or oil agent | | 1.7 | 2.0 |
| Presence/absence of silicone compound | | Absent | Absent |
| Natural origin index (%) | | >99 | >99 |
| Evaluation | Spreadability | C | C |
| result | Powder feeling | D | D |
| | Finish | B | B |
| | Makeup lasting property | A | A |

TABLE 3

| | | Natural | Kinematic | | | | |
|---|---|---|---|---|---|---|---|
| | | origin | viscosity | Example | | | |
| Component (mass %) | | index | (mm²/s) | 6 | 7 | 8 | 9 |
| (a) | Dicaprylyl carbonate *1 | 1.00 | 6 | 26.0 | | 20.0 | |
| | Coco-caprylate/caprate *2 | 1.00 | 6 | | | | 20.0 |
| | Ethyl isostearate *3 | 1.00 | 8 | | | | |
| | Hexyl laurate *4 | 1.00 | 5 | | 25.5 | | |
| | Liquid paraffin *5 | 0.00 | 11 | | | | |
| Oil | Squalane *6 | 1.00 | 30 | | | | |
| agent | Jojoba oil *7 | 1.00 | 40 | | | | |
| (b) | Cellulose powder (14 μm) *8 | 1.00 | | 35.5 | | 49.5 | 58.0 |
| | Cellulose acetate powder (7 μm) *9 | 1.00 | | | 46.4 | | |
| | Urethane powder (7 μm) *10 | 0.00 | | | | | |
| (c) | Dextrin isostearate *11 | 1.00 | | 4.0 | 4.0 | | 2.4 |
| | Candelilla wax extract *12 | 1.00 | | | | 4.0 | |
| | Trimethylsiloxysilicic acid/dimethicone mixture *13 | 0.00 | Dimethicone: 6 | | | | |
| (d) | Polyglyceryl-2 tetraisostearate-treated titanium dioxide *14 | 0.97 | | 16.0 | 16.0 | 16.0 | 16.0 |
| | Polyglyceryl-2 tetraisostearate-treated red iron oxide *15 | 1.00 | | 1.5 | 1.5 | 1.5 | 1.5 |
| | Polyglyceryl-2 tetraisostearate-treated yellow iron oxide *16 | 1.00 | | 0.5 | 0.5 | 0.5 | 0.5 |
| | Polyglyceryl-2 tetraisostearate-treated black iron oxide *17 | 1.00 | | 0.1 | 0.1 | 0.1 | 0.1 |
| | Silicone-treated titanium dioxide *18 | 0.98 | | | | | |
| | Silicone-treated yellow iron oxide *19 | 0.97 | | | | | |
| | Silicone-treated red iron oxide *20 | 0.97 | | | | | |
| | Silicone-treated black iron oxide *21 | 0.98 | | | | | |
| (e) | Sunflower wax *22 | 1.00 | | 3.0 | 2.0 | 3.0 | 1.0 |
| | Carnauba wax *23 | 1.00 | | 0.5 | | 0.5 | 0.5 |
| | Ceresin *24 | 0.00 | | | | | |
| (f) | Sorbitan sesquioleate *25 | 1.00 | | 1.0 | 1.0 | 1.0 | |
| Others | C9-12 alkane *26 | 1.00 | 1.8 | | 3.0 | | |
| | Isododecane *27 | 0.00 | | | | | |
| | Barium sulfate *28 | 1.00 | | 11.9 | | 3.9 | |
| | Disteardimonium hectorite *29 | 0.98 | | | | | |

| Total | | 100.0 | 100.0 | 100.0 | 100.0 |
|---|---|---|---|---|---|
| Mass ratio of component (b) to component (a) or oil agent | | 1.4 | 1.8 | 2.5 | 2.9 |
| Presence/absence of silicone compound | | Absent | Absent | Absent | Absent |
| Natural origin index (%) | | >99 | >99 | >99 | >99 |
| Evaluation | Spreadability | A | A | A | B |
| result | Powder feeling | A | A | A | A |
| | Finish | A | A | B | A |
| | Makeup lasting property | A | A | B | B |

TABLE 4

| | | Natural | Kinematic | Comparative | |
|---|---|---|---|---|---|
| | | origin | viscosity | Example | |
| Component (mass %) | | index | (mm²/s) | 3 | 4 |
| (a) | Dicaprylyl carbonate *1 | 1.00 | 6 | 34.5 | 14.0 |
| | Coco-caprylate/caprate *2 | 1.00 | 6 | | |
| | Ethyl isostearate *3 | 1.00 | 8 | | |
| | Hexyl laurate *4 | 1.00 | 5 | | |
| | Liquid paraffin *5 | 0.00 | 11 | | |
| Oil | Squalane *6 | 1.00 | 30 | | |
| agent | Jojoba oil *7 | 1.00 | 40 | | |
| (b) | Cellulose powder (14 μm) *8 | 1.00 | | 30.0 | 57.5 |
| | Cellulose acetate powder (7 μm) *9 | 1.00 | | | |
| | Urethane powder (7 μm) *10 | 0.00 | | | |
| (c) | Dextrin isostearate *11 | 1.00 | | 4.0 | 4.0 |
| | Candelilla wax extract *12 | 1.00 | | | |
| | Trimethylsiloxysilicic acid/dimethicone mixture *13 | 0.00 | Dimethicone: 6 | | |
| (d) | Polyglyceryl-2 tetraisostearate-treated titanium dioxide *14 | 0.97 | | 16.0 | 16.0 |
| | Polyglyceryl-2 tetraisostearate-treated red iron oxide *15 | 1.00 | | 1.5 | 1.5 |
| | Polyglyceryl-2 tetraisostearate-treated yellow iron oxide *16 | 1.00 | | 0.5 | 0.5 |
| | Polyglyceryl-2 tetraisostearate-treated black iron oxide *17 | 1.00 | | 0.1 | 0.1 |
| | Silicone-treated titanium dioxide *18 | 0.98 | | | |
| | silicone-treated yellow iron oxide *19 | 0.97 | | | |
| | Silicone-treated red iron oxide *20 | 0.97 | | | |
| | Silicone-treated black iron oxide *21 | 0.98 | | | |
| (e) | Sunflower wax *22 | 1.00 | | 3.0 | 3.0 |
| | Carnauba wax *23 | 1.00 | | 0.5 | 0.5 |
| | Ceresin *24 | 0.00 | | | |
| (f) | Sorbitan sesquioleate *25 | 1.00 | | 1.0 | 1.0 |
| Others | C9-12 alkane *26 | 1.00 | 1.8 | | |
| | Isododecane *27 | 0.00 | | | |
| | Barium sulfate *28 | 1.00 | | 8.9 | 1.9 |
| | Disteardimonium hectorite *29 | 0.98 | | | |

| Total | | 100.0 | 100.0 |
|---|---|---|---|
| Mass ratio of component (b) to component (a) or oil agent | | 0.9 | 4.1 |
| Presence/absence of silicone compound | | Absent | Absent |
| Natural origin index (%) | | >99 | >99 |
| Evaluation | Spreadability | A | C |
| result | Powder feeling | D | A |
| | Finish | B | A |
| | Makeup lasting property | A | D |

TABLE 5

| | | Natural | Kinematic | | | Comparative |
|---|---|---|---|---|---|---|
| | | origin | viscosity | Example | | Example |
| Component (mass %) | | index | (mm²/s) | 10 | 11 | 5 |
| (a) | Dicaprylyl carbonate *1 | 1.00 | 6 | 26.5 | | 31.9 |
| | Coco-caprylate/caprate *2 | 1.00 | 6 | | | |
| | Ethyl isostearate *3 | 1.00 | 8 | | 23.9 | |
| | Hexyl laurate *4 | 1.00 | 5 | | | |
| | Liquid paraffin *5 | 0.00 | 11 | | | |
| Oil | Squalane *8 | 1.00 | 30 | | | |
| agent | Jojoba oil *7 | 1.00 | 40 | | | |
| (b) | Cellulose powder (14 μm) *8 | 1.00 | | | 40.0 | 41.5 |
| | Cellulose acetate powder (7 μm) *9 | 1.00 | | 40.0 | | |
| | Urethane powder (7 μm) *10 | 0.00 | | | | |
| (c) | Dextrin isostearate *11 | 1.00 | | 3.0 | 7.0 | |
| | Candelilla wax extract *12 | 1.00 | | | | |
| | Trimethylsiloxysilicic acid/dimethicone mixture *13 | 0.00 | Dimethicone: 6 | | | |
| (d) | Polyglyceryl-2 tetraisostearate-treated titanium dioxide *14 | 0.97 | | 16.0 | 16.0 | 16.0 |
| | Polyglyceryl-2 tetraisostearate-treated red iron oxide *15 | 1.00 | | 1.5 | 1.5 | 1.5 |
| | Polyglyceryl-2 tetraisostearate-treated yellow iron oxide *16 | 1.00 | | 0.5 | 0.5 | 0.5 |
| | Polyglyceryl-2 tetraisostearate-treated black iron oxide *17 | 1.00 | | 0.1 | 0.1 | 0.1 |
| | Silicone-treated titanium dioxide *18 | 0.98 | | | | |
| | Silicone-treated yellow iron oxide *19 | 0.97 | | | | |
| | Silicone-treated red iron oxide *20 | 0.97 | | | | |
| | Silicone-treated black iron oxide *21 | 0.98 | | | | |
| (e) | Sunflower wax *22 | 1.00 | | 3.0 | 3.0 | 3.0 |
| | Carnauba wax *23 | 1.00 | | 0.5 | | 0.5 |
| | Ceresin *24 | 0.00 | | | | |
| (f) | Sorbitan sesquioleate *25 | 1.00 | | 1.0 | 1.0 | 1.0 |
| Others | C9-12 alkane *26 | 1.00 | 1.8 | | | |
| | Isododecane *27 | 0.00 | | | | |
| | Barium sulfate *28 | 1.00 | | 7.9 | 7.0 | 4.0 |
| | Disteardimonium hectorite *29 | 0.98 | | | | |

| Total | | 100.0 | 100.0 | 100.0 |
|---|---|---|---|---|
| Mass ratio of component (b) to component (a) or oil agent | | 1.5 | 1.7 | 1.3 |
| Presence/absence of silicone compound | | Absent | Absent | Absent |
| Natural origin index (%) | | >99 | >99 | >99 |
| Evaluation | Spreadability | A | A | A |
| result | Powder feeling | A | A | A |
| | Finish | A | A | C |
| | Makeup lasting property | A | A | D |

Details of each component used in Examples 1 to 11 and Comparative Examples 1 to 5 are as follows.
1 Dicaprylyl carbonate: Cetiol CC, manufactured by BASF
2 Coco-caprylate/caprate: Liponate CCC MB, manufactured by Vantage Specialty Ingredients, Inc.
3 Ethyl isostearate: EIS-V, manufactured by KOKYU ALCOHOL KOGYO CO., LTD.
4 Hexyl laurate: KAK HL, manufactured by KOKYU ALCOHOL KOGYO CO., LTD.
5 Liquid paraffin: liquid paraffin No. 50S, manufactured by Sanko Chemical Industry Co., Ltd.
6 Squalane: sugar squalane, manufactured by Nikko Chemicals Co., Ltd.
7 Jojoba seed oil: purified jojoba oil, manufactured by Yokozeki Oil & Fat Industries Co., Ltd.
8 Cellulose powder (volume-weighted mean diameter: 14 μm): CELLULOBEADS D-10, manufactured by DAITO CHEMICAL CO., LTD.
9 Cellulose acetate powder (volume-weighted mean diameter: 7 μm): BELLOCEA S7, manufactured by Daicel Corporation
10 Urethane powder (volume-weighted mean diameter: 7 μm): TP Powder D-800, manufactured by TOSHIKI PIGMENT CO., LTD.
11 Dextrin isostearate: Unifilma HVY, manufactured by Chiba Flour Milling Co., Ltd.
12 Candelilla wax extract: candelilla resin E-1, manufactured by Japan Natural Products Co., Ltd.
13 Trimethylsiloxysilicic acid/dimethicone mixture: KF-7312K (resin component: 60%, dimethicone: 40%), manufactured by Shin-Etsu Chemical Co., Ltd.
14 Polyglyceryl-2 tetraisostearate-treated titanium dioxide: PGQ titanium dioxide R250, manufactured by DAITO CHEMICAL CO., LTD.
15 Polyglyceryl-2 tetraisostearate-treated red iron oxide: PGQ Red No. 211P, manufactured by DAITO CHEMICAL CO., LTD.
16 Polyglyceryl-2 tetraisostearate-treated yellow iron oxide: PGQ Yellow No. 602F, manufactured by DAITO CHEMICAL CO., LTD.
17 Polyglyceryl-2 tetraisostearate-treated black iron oxide: PGQ Black 710P, manufactured by DAITO CHEMICAL CO., LTD.
18 Silicone-treated titanium oxide: SA-titanium CR-50 (100%), manufactured by Miyoshi Kasei, Inc.
19 Silicone-treated yellow iron oxide: SA-Yellow LL-100P (100%), manufactured by Miyoshi Kasei, Inc.
20 Silicone-treated red iron oxide: SA-Red R-516PS (100%), manufactured by Miyoshi Kasei, Inc.
21 Silicone-treated black iron oxide: SA-Black BL-100P (100%), manufactured by Miyoshi Kasei, Inc.
22 Sunflower wax: purified sunflower wax, manufactured by Yokozeki Oil & Fat Industries Co., Ltd.
23 Carnauba wax: carnauba wax No. 1, manufactured by S. KATO & CO.
24 Ceresin: purified ceresin N, manufactured by NIKKO RICA CORPORATION.
25 Sorbitan sesquioleate: RHEODOL AO-15V, manufactured by Kao Corporation
26 C9-12 alkane: Jeechem NDA-LC, manufactured by Jeen International Corporation
27 Isododecane: Marukasol R, manufactured by Maruzen Petrochemical CO., LTD.
28 Barium sulfate: plate-like barium sulfate H-LFM, manufactured by Sakai Chemical Industry Co., Ltd.
29 Disteardimonium hectorite: BENTONE 38, manufactured by

### Elementis

The dimethicone contained in the trimethylsiloxysilicic acid/dimethicone mixture used in Example 4 corresponds to the component (a) because the kinematic viscosity at 25° C. is 6 mm²/s and the boiling point at 1 atm is 290° C. or higher.

As shown in Tables 1 to 5, the oil-based cosmetics of Examples 1 to 11, which contained the components (a) to (d) and in which the mass ratio of the content of the component (b) to the content of the component (a) was 1.0 to 3.5, is excellent in spreadability at the time of application, has a powder feeling that did not feel stickiness after application, and is also excellent in makeup lasting property. On the other hand, the oil-based cosmetics of Comparative Examples 1 and 2, in which an oil agent having a high kinematic viscosity was used instead of the component (a), were inferior in spreadability at the time of application, and did not provide a powder feeling after application. In addition, in the oil-based cosmetic of Comparative Example 3 in which the mass ratio of the content of the component (b) to the content of the component (a) was less than 1.0, a powder feeling was not obtained after application. On the other hand, the oil-based cosmetic of Comparative Example 4, in which the mass ratio of the content of the component (b) to the content of the component (a) was more than 3.5, was inferior in spreadability at the time of application, and was inferior in makeup lasting property. Further, the oil-based cosmetic of Comparative Example 5, which did not contain the component (c), was inferior in finish after application, and was inferior in makeup lasting property.

The oil-based cosmetics of Examples 1 to 4 and 6 to 11 are suitable as a solid type foundation, and the oil-based cosmetic of Example 5 is suitable as a liquid type foundation. For reference, formulation examples other than the foundation are shown in Table 6 below. Numerical values in a column of each component in Table 6 indicate a blending ratio (unit:mass %) of the component, and a blank indicates that the component is not blended. In addition, all of kinematic viscosities in Table 6 are values at 25° C. An oil-based cosmetic of Formulation Example 1 is suitable for eye shadow, an oil-based cosmetic of the Formulation Example 2 is suitable for cheek, and an oil-based cosmetic of Formulation Example 3 is suitable for a base cosmetic.

TABLE 6

| | | Natural | Kinematic | | | |
|---|---|---|---|---|---|---|
| | | origin | viscosity | Formulation Example | | |
| Component (mass %) | | index | (mm²/s) | 1 | 2 | 3 |
| (a) | Dicaprylyl carbonate | 1.00 | 6 | | 31.0 | |
| | Coco-caprylate/caprate | 1.00 | 6 | 35.5 | | 40.0 |
| (b) | Cellulose powder (14 μm) | 1.00 | | 40.0 | 59.0 | 46.0 |
| (c) | Dextrin isostearate | 1.00 | | 1.0 | 1.0 | 5.0 |
| (d) | Polyglyceryl-2 tetraisostearate-treated titanium dioxide | 0.97 | | 4.0 | 3.8 | |
| | Polyglyceryl-2 tetraisostearate-treated red iron oxide | 1.00 | | 1.3 | 1.7 | |
| | Polyglyceryl-2 tetraisostearate-treated yellow iron oxide | 1.00 | | 2.7 | | |
| | Polyglyceryl-2 tetraisostearate-treated black iron oxide | 1.00 | | 0.2 | 0.2 | |
| | Red No. 202 | 0.00 | | | 0.3 | |
| (f) | Pearling agent | 1.00 | | 10.0 | | 5.0 |
| (e) | Sunflower Wax | 1.00 | | 3.0 | 1.0 | 2.0 |
| | Carnauba wax | 1.00 | | 0.5 | 1.0 | 1.0 |
| | Sorbitan sesquioleate | 1.00 | | 1.0 | 1.0 | 1.0 |
| Others | Barium sulfate | 1.00 | | 0.8 | | |

| | | | |
|---|---|---|---|
| Total | 100.0 | 100.0 | 100.0 |
| Mass ratio of component (b) to component (a) | 1.1 | 1.9 | 1.2 |
| Presence/absence of silicone compound | Absent | Absent | Absent |
| Natural origin index (%) | >99 | >99 | >99 |

What is claimed is:

1. An oil-based cosmetic comprising:
(a) a non-volatile oil agent having a kinematic viscosity at 25° C. of 4 mm²/s to 20 mm²/s and a boiling point at 1 atm of 290° C. or higher;
(b) a spherical particle;
(c) a film-forming agent; and
(d) a color material,
wherein a mass ratio of a content of (b) the spherical particle to a content of (a) the non-volatile oil agent being 1.0 to 3.5.

2. The oil-based cosmetic according to claim 1, wherein a content ratio of a volatile liquid having a boiling point at 1 atm of lower than 290° C. is less than 5 mass %.

3. The oil-based cosmetic according to claim 1, wherein a content ratio of (a) the non-volatile oil agent is 15 mass % to 50 mass %.

4. The oil-based cosmetic according to claim 1, wherein a content ratio of (b) the spherical particle is 30 mass % to 65 mass %.

5. The oil-based cosmetic according to claim 1, further comprising: (e) a wax.

6. The oil-based cosmetic according to claim 1, wherein the oil-based cosmetic does not contain a silicone compound.

7. The oil-based cosmetic according to claim 1, wherein a natural origin index (excluding water) calculated in accordance with ISO 16128 is 90% or more.

8. The oil-based cosmetic according to claim 1, which is a cosmetic for foundation, eye shadow, eyebrow, cheek, or base.
